(19) United States
(12) Patent Application Publication
(10) Pub. No.: US 2012/0134878 A1
(43) Pub. Date: May 31, 2012

(54) UNIT AND A METHOD FOR STERILIZING CONTAINER CLOSURES

(75) Inventor: Angelo Silvestri, Parma (IT)

(73) Assignee: SIDEL S.P.A. Con Socio Unico, Parma (IT)

(21) Appl. No.: 13/319,055

(22) PCT Filed: May 5, 2009
(86) PCT No.: PCT/IT2009/000203

§ 371 Date: Feb. 16, 2012

(51) Int. Cl. A61L 2/08

(52) U.S. Cl. 422/22; 422/292

(57) ABSTRACT

There is described a unit for sterilizing container closures comprising: a process chamber having an inlet for receiving a succession of closures to be sterilized and an outlet from which sterilized closures exit; conveying means for advancing the closures through the process chamber along a predetermined path (P); and radiation emitting means acting inside the process chamber, facing the closures moving along the above path (P) and which can be activated for directing sterilizing radiations on said closures to sterilize their surfaces; the conveying means comprise actuator means acting on each closure to produce a rolling movement thereof while it advances along its path (P).

### TECHNICAL FIELD

The present invention relates to a unit and a method for sterilizing closures, in particular cylindrical screw caps, designed to be fitted onto respective bottles or containers, in particular of the type filled with liquid or powder products when it is appropriate or necessary to maintain aseptic and/or ultra clean conditions.

### BACKGROUND ART

As it is commonly known, microbiological decontamination of the materials used for packaging some particular products, such as food products (for instance milk, fruit juices, beverages, etc.), is normally required in order to guarantee the quality and the shelf-life of such products.

Sterilizing operations are therefore normally performed on both the containers and the closures thereof in order to destroy bacteria, moulds, viruses, and other microorganisms.

Typically, the materials to decontaminate are first immersed in a bath of, or sprayed with, a liquid sterilizing agent for a predetermined time to ensure a complete treatment, then withdrawn from the bath or from the treatment compartment and finally subjected to a drying operation, e.g. by means of hot-air jets or to a rinsing phase with sterile water, in order to remove any residual sterilizing agent. It is pointed out that the amount of sterilizing agent allowed in the packaged product is governed by strict standards (the maximum permissible amount being in the order of a fraction of one part per million).

Particularly in the case of plastic materials, such as the ones typically employed for container closures, the air conventionally used for removing the residual sterilizing agent cannot be heated to a high temperature to avoid the likelihood of deforming the treated materials. Therefore, this operation normally has a very long duration in order to ensure adherence to the above-mentioned standards.

Besides, the containers closures have some internal surfaces, such as threads, ribs and so on, forming recesses in which residual sterilizing agent may become trapped, and from which complete removal of the sterilizing agent can be achieved with extreme difficulty.

### DISCLOSURE OF INVENTION

It is an object of the present invention to provide a unit for sterilizing closures for containers, designed to overcome the above drawbacks in a straightforward and low-cost manner.

This object is achieved by a unit for sterilizing closures for containers, as claimed in claim 1.

The present invention also relates to a method for sterilizing closures for containers, as claimed in claim 13.

### BRIEF DESCRIPTION OF THE DRAWINGS

Two preferred, non-limiting embodiments of the present invention will be described by way of example with reference to the accompanying drawings, in which:

FIG. 1 shows a view in perspective of a container closure sterilizing unit in accordance with the teachings of the present invention;

FIG. 2 shows a larger-scale section view of a container closure processed in the FIG. 1 sterilizing unit;

FIG. 3 shows a top plan view of the FIG. 1 sterilizing unit;

FIG. 4 shows a larger-scale view in perspective of the FIG. 1 sterilizing unit, in an open condition;

FIG. 5 shows a larger-scale front view, with parts removed for clarity, of an inner portion of the FIG. 4 sterilizing unit; and

FIG. 6 shows a top plan view of a container closure sterilizing unit in accordance with a different embodiment of the present invention.

### BEST MODE FOR CARRYING OUT THE INVENTION

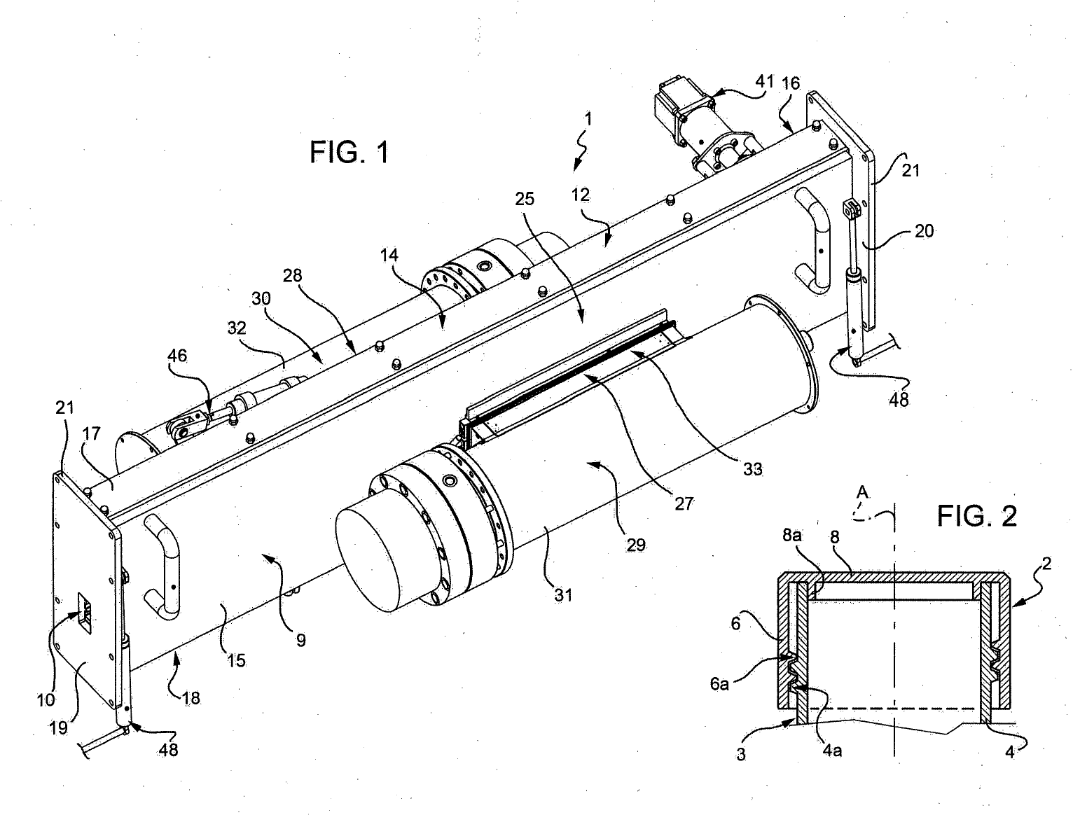

Number 1 in FIGS. 1, 3 and 4 indicates as a whole a unit for sterilizing closures 2 designed to be fitted onto respective bottles or containers 3 (only partially visible in FIG. 2), in particular of the type filled with liquid or powder products, such as pourable food products.

Unit 1 is adapted to be integrated into plants (not shown) for handling containers 3 in order to fill them with the liquid or powder products and to close them with the respective closures 2.

In the example shown (see in particular FIG. 2), the closures 2 processed by sterilizing unit 1 are cylindrical screw caps adapted to be fitted onto cylindrical necks 4 of respective containers 3 having an external thread 4a. More specifically, each closure 2 has an axis A and comprises a cylindrical side wall 6 provided with an internal thread 6a to be engaged with the complementary thread 4a of the relative container neck 4, and a disk-shaped top wall 8 peripherally integral with side wall 6 and adapted to close, in use, the container neck 4.

Disk-shaped top wall 8 is also provided with one annular sealing rib 8a on the side destined in use to cooperate with necks 4 of containers 3; annular rib 8a typically has the function to ensure sealing and resealing of containers 3 after the first opening. Other ribs or projecting elements can be present on the closure, either for technical or for aesthetical purpose.

With reference to FIGS. 1, 3, 4 and 5, unit 1 basically comprises a process chamber 9 having an inlet 10, for receiving a succession of closures 2 to be sterilized, and an outlet 11, from which sterilized closures 2 exit, sterilizing means 12 acting inside process chamber 9, and conveying means 13 for advancing closures 2 through process chamber 9 along a predetermined path P, in the specific case defined by a horizontal straight line.

Process chamber 9 is delimited by a box-type structure 14 having, in the example shown, a substantially parallelepiped shape.

In particular, box-type structure 14 comprises a front and a rear vertical wall 15, 16, extending parallel to, and on opposite sides of, path P; a top and a bottom horizontal wall 17, 18, orthogonal to walls 15, 16 and parallel to path P; and a pair of side walls 19, 20 orthogonal to walls 15-18 and path P.

More specifically, front and rear walls 15, 16 have a length corresponding to the extension of path P, whilst side walls 19, 20 define, in a direction orthogonal to path P and parallel to walls 17, 18, the thickness of box-type structure 14, which is reduced with respect to the other sizes.

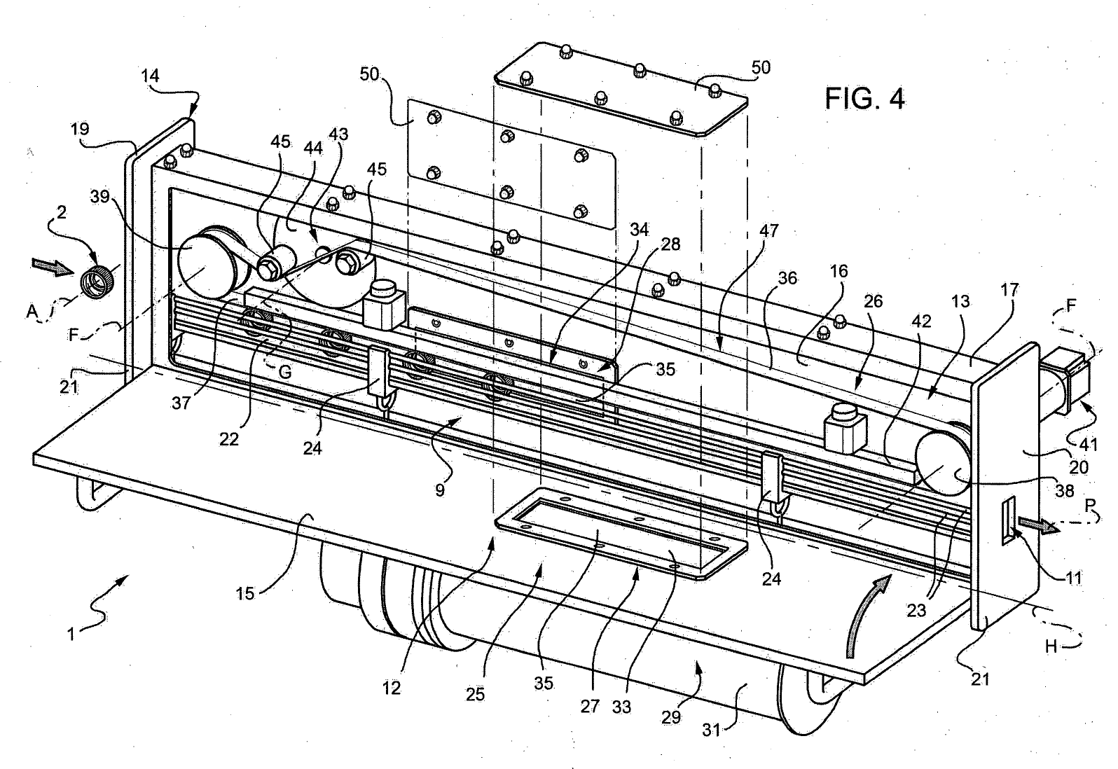

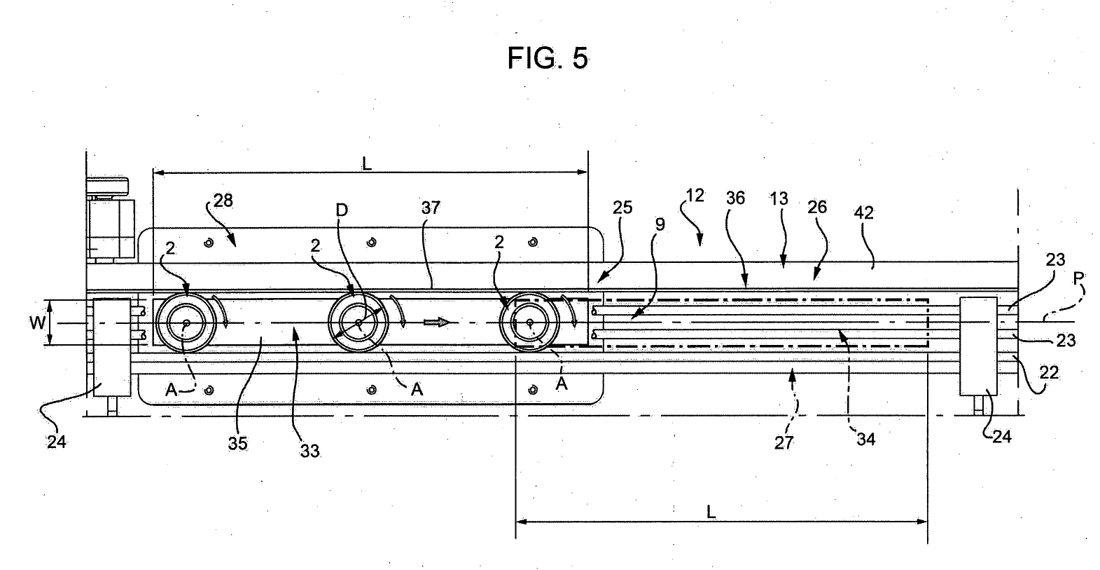

As shown in FIGS. 1, 3 and 4, side walls 19, 20 protrude externally from the overall profile of walls 15-18 so as to define a rectangular peripheral strip portion adapted to be secured to the supporting frame (not shown) of the container handling plant. Inlet 10 and outlet 11 are defined by relative rectangular openings provided into side walls 19, 20, respectively. More precisely, inlet 10 and outlet 11 have sizes suitable to allow passage of one closure 2 at any one time in a vertically-oriented position (FIGS. 4 and 5), in which axis A of each closure 2 is orthogonal to path P and to front and rear wall 15, 16; in other words, in the vertically-oriented position of closures 2, disk-shaped top wall 8 extends parallel to front and rear wall 15, 16.

In the example shown, closures 2 enter box-type structure 14 with their top walls 8 closer to rear wall than front wall 15, and are moved inside process chamber 9 on a horizontal supporting surface 22 parallel to path P.

According to a preferred embodiment of the present invention, supporting surface 22 is defined by the top of a plate fixed to the box-type structure 14 on which closures 2 move under the thrust of conveying means 13, as better explained later on.

Entry of closures 2 into box-type structure 14 is controlled by a push device, such as an air blower (not shown), which acts on one closure 2 at any one time; in this way, it is possible to space out closures 2 when they enter process chamber 9.

Closures 2 are maintained in the vertically-oriented position inside process chamber 9 by two series of longitudinal horizontal rails 23 arranged on both sides of path P and supported by vertical brackets 24 secured to supporting surface 22.

Advantageously, sterilizing means 12 comprise radiation emitting means 25 facing the closures 2 moving along path P and which can be activated for directing surface sterilizing radiations on said closures.

The peculiarity of this kind of sterilizing means is the fact that sterilization can be achieved only on the irradiated parts of the surfaces to be treated.

In order to avoid one or more portions of the surfaces of closures 2 being in shadow with respect to radiation emitting means 25, conveying means 13 comprise actuator means 26 acting on each closure 2 to produce simultaneously both an advancing of said closure 2 along path P and a rolling movement thereof about axis A. In this way, complete irradiation of any area of closures 2 can be achieved.

According to a preferred embodiment of the present invention, radiation emitting means 25 comprise a pair of electron beam emitters 27, 28, respectively fitted to front and rear wall 15, 16 of box-type structure 14 for directing respective electron beams, having an energy at most equal to 200 KeV, onto opposite faces of closures 2 advancing along path P.

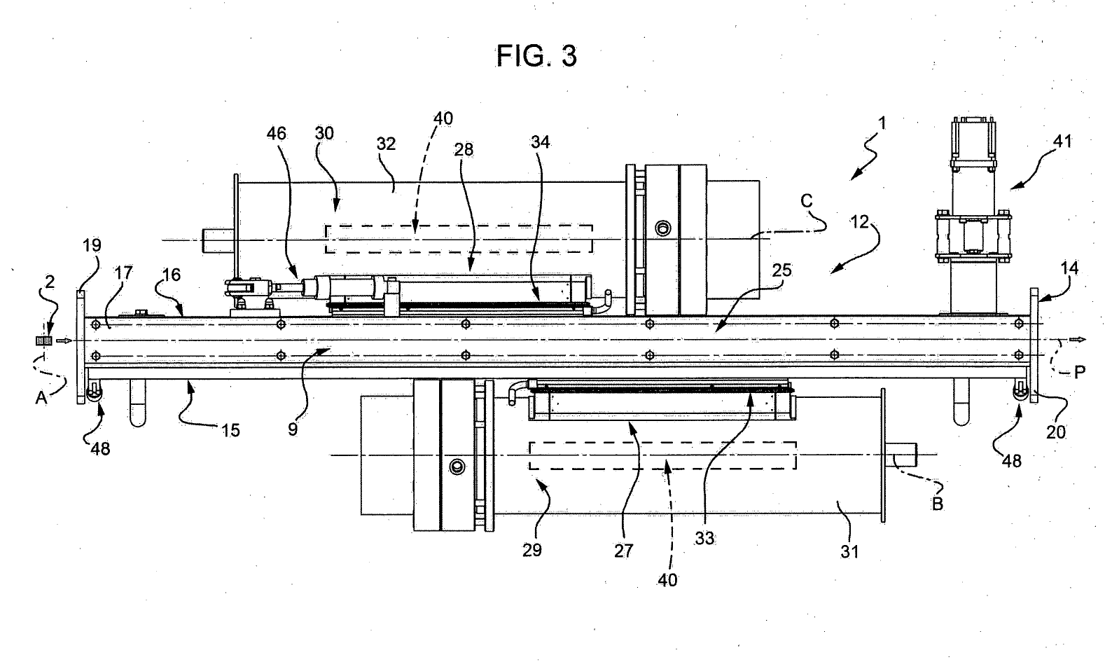

In particular, each emitter 27, 28 comprises a vacuum chamber 29, 30 and an electron generator 40 (only schematically shown in FIG. 3), such as a tungsten element, positioned therein and heated for generating electrons. It is clear that any other electron generating means may be used.

In the example shown, each vacuum chamber 29, 30 is incorporated in a relative tubular housing 31, 32, externally fastened to a relative wall 15, 16 of box-type structure 14 and having an axis B, C parallel to path P and walls 15-18.

Vacuum chambers 29, 30 communicate with process chamber 9 through relative windows 33, 34, respectively provided in front and rear wall 15, 16 of box-type structure 14, facing closures 2 while moving along path P and each closed by a relative window foil 35, which can be easily penetrated by electrons.

In practice, electron beams are emitted from each window 33, 34 towards the facing closures 2 advancing along path P. In order to ensure maximum surface coverage of closures 2, specific reflectors can be provided (known per se and not shown) to generate multidirectional radiation emission from each window 33, 34.

Window foil 35 is formed from a high strength metallic material, such as titanium, in order to withstand the pressure differential between process chamber 9 (kept in low overpressure) and the interior of the relative vacuum chamber 29, 30.

With particular reference to FIGS. 4 and 5, each window 33, 34 has a rectangular profile with a length L parallel to path P and a width W orthogonal to path P and to axes A of closures 2 advancing through process chamber 9.

Advantageously, the width W of each window 33, 34 can be smaller than the external diameter D of closures 2. In this way, thanks to the rolling movement imposed to closures 2, it is possible to obtain the following results:
any external surface of closures 2 is irradiated and therefore sterilized; and
the quantity of energy transferred to each closure 2 is maximised as it is concentrated on a reduced area (windows 33, 34) with respect to the closure diameter D.

It is, in fact, commonly known that the quantity of energy transferred through electron beams is in inverse proportion to the dimensions of the window on which said electron beams are directed.

Advantageously, as clearly shown in FIGS. 4 and 5 (wherein the profile of window 33 is schematically indicated with a dash-to-point line), in order to avoid any risk of overheating closures 2 and the zones of box-type structure 14 subjected to electron beams, windows 33, 34 are at least partially offset with respect to each other in a direction parallel to axes A of closures 2 in the vertically-oriented position. In particular, as disclosed in FIG. 5, only a little overlap is foreseen between windows 33 and 34.

With reference to FIG. 4, conveying means 13 comprise a driving element 47, which, in a preferred embodiment, comprises a powered endless belt 36 having an active portion 37 parallel to, and spaced from, supporting surface 22 and acting on the side of each closure 2 opposite the one resting on supporting surface 22.

In particular, belt 36 is wound around a pair of pulleys 38, 39, having respective axes F parallel to axes A of closures 2 advancing along path P; more specifically, one of the pulleys 38 is fitted onto an output shaft of an electric motor unit 41 and drives belt 36, whilst the other one 39 is driven by the latter.

Active portion 37 of belt 36 slides along and under a longitudinal guide bar 42 affixed to box-type structure 14 in a position parallel to, and spaced from, supporting surface 22.

A tightener 43 is also provided to adjust belt tension; in the example shown, tightener 43 includes a disk-shaped member 44 fitted to rear wall 16 of box-type structure 14 in a rotating manner about an axis G parallel to axes A of closures 2 as well as to axes F of the pulleys 38, 39, a pair of wheels 45 on which belt 36 is partially wound and which project from diametrically opposed portions of a peripheral zone of disk-shaped member 44 towards the inside of process chamber 9, and an actuator member 46, preferably a pneumatic cylinder, acting on disk-shaped member 44 to rotate it about its axis G in order to change the relative positions of wheels 45 and to increase or decrease tension of belt 36.

In view of the above, powered belt 36 defines a positive transport system to advance closures 2 along supporting surface 22 through a rolling movement about their axes A.

With particular reference to FIG. 4, front wall 15 of box-type structure 14 is hinged to bottom wall 18 about an axis H parallel to path P so as to allow opening of this structure for maintenance or in case of any malfunction. As shown in FIG. 4, front wall 15 can be rotated about hinge axis H to reach a substantially horizontal position. A pair of air springs 48 (FIGS. 1 and 3) allow to slow down the opening movement of front wall 15, which is clearly subjected to the weight of emitter 27 secured thereon.

Box-type structure 14 is periodically subjected to washing cycles with detergent liquids at high pressure, such as 20 bar; in this case, in order to avoid breaking of window foils 35, a cover plate 50 (FIG. 4) is fitted to each window 33, 34 to protect it.

In use, one closure 2 at any one time is blown through inlet 10 so entering box-type structure 14 and therefore process chamber 9; in this way, closures 2 reach path P at different time intervals so being spaced a predetermined distance apart.

Each closure 2 is then advanced along supporting surface 22 by active portion 37 of powered belt 36; in particular, belt 36 cooperates with side wall 6 of each closure on a portion thereof opposite the one contacting supporting surface 22. The difference of speed between belt 36 and supporting surface 22 produces an advancing of closures 2 along path P through a rolling movement thereof about their axes A.

Closures 2 are maintained in the vertically-oriented position while advancing along path P by longitudinal horizontal rails 23.

In the meantime, the electrons are vacuum accelerated into beams on the inside of tubular housings 31, 32 by respective electric fields generated by potential differences between the electron generators 40 and the respective window foils 35.

The electrons reach their maximum speed inside the vacuum environment and decelerate and gradually lose part of their energy on colliding with the atoms constituting window foils 35 and closures 2.

In the example shown, the energy produced by the electron beams striking closures 2, which are moving along path P, kills any microorganisms in the closure surfaces.

Thanks to the rolling movement imposed to closures 2 by belt 36, any portion of the external surfaces of closures 2 is irradiated.

In the example shown, sterilization occurs first on the external side of closures 2 through window 34 and then on the internal side thereof (including thread 6a and annular rib 8a) through window 33.

Given their low energy level (at most equal to 200 KeV), the electron beams coming out of emitters 27, 28 penetrate respective opposite faces of closures 2 to a depth of a few μm, which is sufficient to ensure complete surface sterilization thereof.

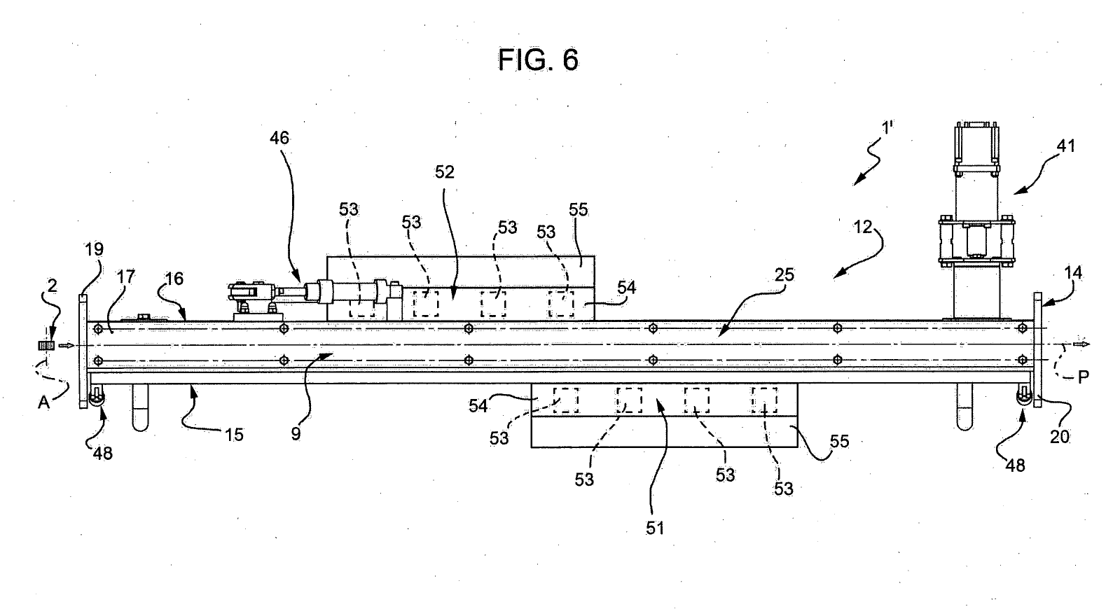

Number 1′ in FIG. 6 indicates as a whole a container closure sterilizing unit in accordance with a different embodiment of the present invention.

Sterilizing unit 1′ being similar to unit 1, the following description is limited to the differences between the two, and using the same reference numbers, where possible, for identical or corresponding parts of units 1 and 1′.

In particular, unit 1′ differs from unit 1 in that radiation emitting means 25 comprise a pair of pulsed light emitters 51, 52 (only schematically shown in FIG. 6), which are arranged on opposite sides of path P and which can be activated to direct respective intense luminous flashes onto opposite faces of the advancing closures 2.

More specifically, each emitter 51, 52 comprises one or more arc lamps 53 functioning in pulse mode and arranged on a relative side of path P along a direction parallel thereto, and a reflector 54 to direct and concentrate the light towards the zone in which closures 2 under treatment pass.

In this case, the sterilization is based on the bactericidal effect of ultraviolet rays contained in the intense flashes of white light emitted by lamps 53.

The energy necessary for the closure decontamination performed by each emitter 51, 52 is accumulated for a short period in a capacitor 55; a high voltage signal sparks arc formation and the liberation of the electrical energy in the relative lamp 53, which is converted into luminous energy. In practice, each lamp 53 contains a ionized gas, such as Xenon, whose ionization is increased by the electric current generated by the above-mentioned high voltage signal; this activates light emission.

The advantages of sterilizing units 1, 1′ and the relative sterilizing methods according to the present invention will be clear from the above description.

In particular, thanks to the fact that closures 2 are sterilized by irradiation instead of being first immersed in a liquid sterilizing agent and then dried, it is possible to achieve the following results:
no residue of sterilizing agent remains on the processed closures after the complete treatment; and
no additional means are required for removing from the processed closures the sterilizing agent normally used in known units of the type described previously;
no water consumption is necessary;
no chemical consumption is necessary;
no chemical emissions through exhausts occur.

Moreover, thanks to the fact that closures 2 roll while advancing in front of radiation emitting means 25, any surface or irregularity of the closures may be reached.

Besides, the use of low-voltage electron beams or pulsed light or any other kind of surface sterilizing radiations allows to obtain a decontaminating effect with no penetration or with a very reduced penetration (of a few μm) of these radiations into the treated material, so minimizing any possible alteration thereof and preventing closures 2 from acquiring an unpleasant taste which may be transmitted to the food product.

Furthermore, in the case of low-voltage electron beams, the rolling movement imparted to closures 2 inside process chamber 9 allows to use emitting windows 33, 34 of reduced sizes (in particular having a width W smaller than the external diameter of the treated closures), so maximizing the quantity of energy transferred to each closure 2 without impairing the effectiveness of the sterilizing treatment.

Clearly, changes may be made to units 1, 1′ and to the method as described and illustrated herein without, however, departing from the scope of protection as defined in the accompanying claims.

In particular, the rolling movement of closures 2 may also be obtained by imparting different speeds to belt 36 and supporting surface 22 or even by moving supporting surface 22 in a direction opposite the one of belt 36; the only condition to have an advancing of closures 2 along path P is that the speed of belt 36 is bigger than the one of supporting surface 22.

1. A unit for sterilizing closures for containers comprising:
a process chamber having an inlet for receiving a succession of closures to be sterilized and an outlet from which sterilized closures exit;
sterilizing means acting inside said process chamber; and
conveying means for advancing said closures through the process chamber along a predetermined path (P);
wherein said sterilizing means comprise radiation emitting means facing the closures moving along said path (P) and which can be activated for directing sterilizing radiations on the said closures, and wherein said conveying means comprise actuator means acting on each closure to produce a rolling movement of said closure while advancing along said path (P).

2. A unit as claimed in claim 1 for sterilizing closures having an axis (A), wherein each closure is advanced by said conveying means through said process chamber with its axis (A) transversal to said path (P), and wherein said rolling movement of each closure is produced by said actuator means about said axis (A).

3. A unit as claimed in claim 1, wherein said conveying means comprise a supporting surface on which said closures roll as a result of the action produced by said actuator means.

4. A unit as claimed in claim 3, wherein said actuator means comprise a driving element configured to cooperate with each closure on the side thereof opposite the one resting on the supporting surface, wherein the actuator means if for moving the driving element at a predetermined relative speed with respect to said supporting surface along said path (P).

5. A unit as claimed in claim 3, wherein said supporting surface is fixed.

6. A unit as claimed in claim 4, wherein said driving element comprises a powered endless belt having an active portion parallel to, and spaced from, said supporting surface and configured to act on said closures.

7. A unit as claimed in claim 1, wherein said radiation emitting means comprise a pair of electron beam emitters arranged on opposite sides of said path (P) configured to direct respective electron beams, having an energy of at most 200 KeV, onto opposite faces of the advancing closures.

8. A unit as claimed in claim 7, wherein each electron beam emitter comprises a relative vacuum chamber and a relative electron generator positioned therein, and wherein each vacuum chamber is configured to communicate with said process chamber through a relative window, from which electron beams are emitted towards the facing closures advancing along said path (P).

9. A unit as claimed in claim 8, wherein each window has a longitudinal size (L) parallel to said path (P) and a transversal size (W) orthogonal to said path (P) and to the axes (A) of the closures, and wherein said transversal size (W) is smaller than the external diameter of said closures.

10. A unit as claimed in claim 8, wherein said windows of said emitters, arranged on opposite sides of said path (P), are at least partially offset with respect to each other in a direction parallel to the axes (A) of the closures advancing through said process chamber.

11. A unit as claimed in claim 1, wherein said process chamber is delimited by a box-type structure having at least one hinged wall, which can be opened for allowing maintenance or repairs in case of malfunction.

12. A unit as claimed in claim 1, wherein said radiation emitting means comprise a pair of pulsed light emitters arranged on opposite sides of said path (P) and directing respective intense luminous flashes onto opposite faces of the advancing closures.

13. A method for sterilizing closures for containers comprising the steps of:
feeding a succession of closures to be sterilized to an inlet of a process chamber;
advancing said closures through the process chamber along a predetermined path (P) towards an outlet of said process chamber;
sterilizing said closures while advancing inside said process chamber;
wherein sterilizing comprises the step of directing sterilizing radiations on said closures while advancing along said path (P), and in that said step of advancing comprises the step of producing a rolling movement of said closures along said path (P).

14. A method as claimed in claim 13 for sterilizing closures having an axis (A), wherein each closure is advanced through said process chamber with its axis (A) transversal to said path (P), and wherein said rolling movement of each closure is produced about said axis (A).

15. A method as claimed in claim 13, wherein said sterilizing radiations comprise emitting electron beams having an energy of at most 200 KeV and directed onto opposite faces of the advancing closures.

16. A method as claimed in claim 15, wherein said electron beams are emitted from respective windows of said process chamber arranged on opposite sides of said path (P).

17. A method as claimed in claim 16, wherein the external diameter of the closures under treatment is bigger than a transversal size (W) of said windows, measured in a direction orthogonal to said path (P) and to the axes (A) of said closures.

18. A method as claimed in claim 16, wherein said windows are at least partially offset with respect to each other in a direction parallel to the axes (A) of the closures advancing through said process chamber.

19. A method as claimed in claim 13, wherein said sterilizing radiations comprise pulsed light radiations directed onto opposite faces of the advancing closures.

20. A unit for sterilizing closures for containers, comprising:
a process chamber having an inlet configured to receive a succession of closures to be sterilized and an outlet for the sterilized closures;
a sterilizer configured to act inside said process chamber; and
a conveyor configure to advance said closures through the process chamber along a predetermined path (P);
wherein said sterilizer comprises a radiation emitter configured to face one or more of the closures along said path (P) and to direct sterilizing radiations on the said closures, and wherein said conveyer includes an actuator configured to act on each closure to produce a rolling movement of said closure hile advancing along said path (P).
